(19) United States
(12) Patent Application Publication
(10) Pub. No.: US 2023/0338178 A1
(43) Pub. Date: Oct. 26, 2023

(54) UNWEIGHTING LEG ORTHOTIC SYSTEM

(71) Applicant: Rod Montanio, Simi Valley, CA (US)

(72) Inventor: Rod Montanio, Simi Valley, CA (US)

(21) Appl. No.: 18/136,534

(22) Filed: Apr. 19, 2023

Related U.S. Application Data
(60) Provisional application No. 63/332,802, filed on Apr. 20, 2022.

(51) Int. Cl. A61F 5/01

(52) U.S. Cl. CPC A61F 5/0123; A61F 5/0111

(57) ABSTRACT

A leg orthotic is provided. The orthotic includes a frame fixed to a user's lower limb by various wrappings. The user's weight is distributed such that their foot floats freely within worn orthotic. The user's limb may be insulated from the frame by various protective sleeve-like elements or padding. The orthotic may be fit with an optional waist modification or thoracic modification and be worn over clothing. The orthotic generally, and especially lower components, are optionally removable for convenience or ease of application.

### RELATED APPLICATIONS

This patent application relates to and claims priority from U.S. Provisional Patent Application No. 63/332,802 entitled “FLOATING SUPPORT LEG BRACES” filed Apr. 20, 2022 and is incorporated herein by reference including its specification.

### FIELD OF THE INVENTION

Embodiments of the disclosure relate generally to musculoskeletal braces. More particularly embodiments of the present disclosure relate to unweighting leg orthotics.

### BACKGROUND OF THE INVENTION

Traditional leg braces focus support on primary joints, i.e., knees or ankles, with discrete points of contact at certain locations along the legs. Such configurations create pressure or “pinch” points resulting in discomfort for the user. Thus, such points of contact make the use of such braces—whether prolonged or short-term—difficult, painful, or even injurious, depending on intensity of use.

Many people that use leg braces or other walking supports often do so due to inability to bear any weight upon the soles of their feet. This may be due to diabetic ulcers, radial neuropathy or similar injury, or simply because the foot is unable to bear the weight along the axis of the leg bones. Existing leg braces are unable to provide adequate and comfortable support whereby a user can stand or walk without bearing their body weight upon the soles of their feet.

Accordingly, there is a need for an improved supporting leg brace that addresses these disadvantages. The present invention fulfills these needs and provides other related advantages.

### SUMMARY OF THE INVENTION

The present disclosure is directed to a novel leg orthotic system that provides improved support to an ambulating or orthostatic person. The orthotic may aid people having weakened or otherwise compromised lower limbs who may experience discomfort, pain, or be unable to stand while bearing weight on one or both of their feet.

The leg orthotic provides substantially full-area support along a person's leg such that the leg is comfortably supported without any areas of focused pressure or pinch. When properly configured the leg orthotic provides adequate support such that the sole of the person's foot is virtually “floating” above whatever floor surface over which the user is positioned. The supports as described below can comfortably support the user's body weight. The weight is transferred to the ground through the leg orthotic supports such that a user's weight is not borne by the soles of the user's feet.

In one embodiment, the inventive leg orthotic has a leg sleeve—similar to a leg on a wetsuit—configured to extend from proximate to a person's ankle to proximate to a person's hip. The leg sleeve is preferably made from neoprene or similar material to provide a snug but comfortable and breathable fit along substantially the entire length of a person's leg. The leg sleeve is similar to one that might be found in a wetsuit or similar article. The leg sleeve may be made from neoprene, lycra, thermocline, silicone rubber, or similar rubber materials that provide comparable snugness, support, protection, and durability. The leg sleeve may be configured for different thicknesses and weights provided that the material can afford adequate support and protection when used as described below.

Where the inventive leg orthotic is configured for use on both legs simultaneously, the leg sleeve may be configured as separate sleeves for each leg or as a pair of pants that include coverage for the hips, groin, gluteal, and waist areas. The leg sleeves may have an enclosed configuration such that they are placed on a user's legs by sliding up from the feet akin to donning a pair of pants. Alternatively, the leg sleeves may include a fastener along its length such that the leg sleeve may be substantially opened and wrapped around the user's leg then the fastener closed.

Whether for one or both legs, each leg sleeve preferably includes rigid or semi-rigid supports attached along the length of both the inseam and the outseam of the leg sleeve. The rigid or semi-rigid supports are attached to the leg sleeve at every point from the ankle to the hip/groin. The rigid supports preferably have hinges or similar flexion joints proximate to the user's knees. The rigid supports may also be contoured to follow the shape of the user's legs. Because of the material of the leg sleeve and the attachment of the support to the leg sleeve, it is not necessary to have additional straps or fasteners along the length of the leg sleeve. However, it may be advantageous to include straps and fasteners that encircle the user's leg and supports proximate to the hips and ankles, as well as around the knee —above, below, or both.

The lowest end of the rigid or semi-rigid supports, proximate to the user's ankles, are preferably attached to a frame that surrounds each foot. The frame provides a rigid structure around the foot and has sufficient height such that when the lower ends of the supports are affixed thereto and the leg sleeves support the user's weight, the soles of the user's feet “float” over the floor or surface upon which the foot frame rests. Between the rigid supports and the foot frame may be included a spring or other shock absorbing structure to increase the user's comfort.

The connection between the rigid supports and the foot frame is preferably hinged to provide additional comfort to the user. Within the foot frame is preferably an inverted Y-shaped bracket or similar connection between the end of the rigid support and at least two points along the bottom edge of the foot frame. The at least two points along the bottom edge of the foot frame are preferably configured to provide a wide base of support proximate to the heel and the ball of the user's feet. The bottom edge of the foot frame may also be contoured to provide some similarity to the manner in which the sole of a shoe may rock or pivot on a surface.

In preferred embodiments, a series of pads attached to the leg orthotic obviates the need for such sleeves. The pads may be comprised of the same or similar materials as the sleeves.

An unweighting leg orthotic system may comprise a set of upper supports hingedly coupled to a set of lower supports at a set of joints. Embodiments may have a plurality of pads configured for removable attachment to the user's leg. The set of joints may have a medial joint face, a lateral joint face, and a set of joint pads associated therewith configured so as to abut against the user's knee.

The unweighting leg orthotic (ULO) may comprise an upper medial member and an upper lateral member associated with said set of upper supports. It may also have a lower medial member and a lower lateral member associated with the set of lower supports.

The ULO may have a pedal protector removably connected to the set of lower supports.

The pedal protector may have an insole further comprising a toe bolster for maintaining both ankle flexion and an open gap between the insole of the pedal protector and sole of the user's foot. There may be a quick release interface between the set of lower supports and the pedal protector. The set of lower supports may be adjustable in length so that a user may vary the length of a gap between the insole of the pedal protector and the user's foot.

The ULO pedal protector may be configured for various sporting contexts. The protector may comprise a boot compatible with snowboard bindings, ski bindings, waterski bindings, skateboard bindings and scooter bindings, among others.

The ULO may have an assortment of wraps to facilitate binding to a user's leg. A plurality of upper wraps may engage with the upper member and upper lateral member. The wraps may be configured so as to removably hold the upper members securely against corresponding medial faces and lateral faces of the user's leg.

A plurality of lower wraps may engage with the lower medial member and lower lateral member of the ULO. The wraps may be configured so as to removably hold the lower members securely against a corresponding medial face and lateral face of the user's leg.

The plurality of upper wraps and plurality of lower wraps may each comprise a medial wrap end and a lateral wrap end. They may further comprise a fastener configured to removably connect the lateral wrap end to the medial wrap end.

The ULO may be equipped with various pads. A plurality of upper pads may be associated with the upper medial member and upper lateral member. The upper pads are configured to abut a substantial length of the corresponding medial and lateral face of the user's upper leg. The ULO may further comprise a plurality of lower pads associated with the lower medial member and lower lateral member of the device. The plurality of lower pads is configured so as to abut a substantial length of the corresponding medial and lateral face of a lower portion of the user's leg.

The ULO may also have a pedal balancer. The balancer may be configured for use on a user's foot opposite the leg brace. The pedal balancer has a sole thickness equal in dimension to the combined dimension of the gap. It has a sole thickness corresponding to the combined thickness of the open gap and insole of the pedal protector.

Multiple ULOs may be used in concert, or duplicate. In such configurations a first brace and a second brace, each a ULO, may be equipped. Tandem ULOs have substantially the same features as single braces and may be configured for a first hip extension and second hip extension enabling an optional waist band and thoracic girdle.

A ULO may have a first hip extension hingedly associated with the upper lateral member of the first leg brace. A second hip extension may be hingedly associated with the upper lateral member of the second leg brace. A first hip pad associates with the first hip extension and a second hip pad associates with the second hip extension. The waist band associates with the extensions and is configured to traverse a user's waist. The waist band also removably holds the hip pads securely against the corresponding lateral faces of the user's waist.

Other ULO embodiments have a thoracic girdle. The girdle may comprise a first thoracic extension and second thoracic extension that associates with the hip extensions. The thoracic girdle generally transverses the user's torso. It removably holds each thoracic extension against corresponding lateral sides of the user's torso. The girdle further comprises a first thoracic pad coupled to a first thoracic extension and a second thoracic pad coupled to a second thoracic extension. In addition, the thoracic extensions may be coupled to a first axillary support and a second axillary support. The axillary supports are adjustable relative to corresponding first and second thoracic extension.

Each embodiment of the unweighting leg orthotic may be configured to generally support all of a user's weight in the leg brace. The plurality of upper wraps and lower wraps engaging with the various members configure the ULO to support substantially all of the user's weight in the leg brace when removably held against the corresponding medial face and lateral faces of the user's leg.

Other features and advantages of the present invention will become apparent from the following more detailed description, taken in conjunction with the accompanying drawings, which illustrate, by way of example, the principles of the invention.

### BRIEF DESCRIPTION OF THE FIGURES

These and other features and advantages of the present invention will become appreciated, as the same becomes better understood with reference to the specification, claims and drawings herein:

FIG. 1 is a perspective view of a preferred embodiment of an unweighted leg orthotic system of the present invention;

FIG. 2 is a front view of the orthotic of FIG. 1;

FIG. 3 is a lateral view of the orthotic of FIG. 1;

FIG. 4 is an exploded perspective view of the orthotic of FIG. 1;

FIG. 5 is a zoom view of the lower support members of the orthotic of FIG. 1;

FIG. 6 is a lateral environmental view of the orthotic of FIG. 1 showing a fully assembled embodiment applied to a user's leg with outer wraps;

FIG. 7 is a medial zoom view of the orthotic of FIG. 1 showing outer wraps;

FIG. 8 is a front zoom view of the orthotic of FIG. 1 showing lower support members;

FIG. 9 is a perspective zoom environmental view of the orthotic of FIG. 1 showing an assembled pedal protector;

FIG. 10 is a partially exploded zoom view of the orthotic of FIG. 1 showing detachable features of the lower support members and pedal protector;

FIG. 11 is a sectional view across a midline of an embodiment of the orthotic of FIG. 1 showing a user's unweighted foot;

FIG. 12 is a lateral view of the orthotic of FIG. 1 showing an optional flat sole;

FIG. 13 is a lateral view of the orthotic of FIG. 1 showing an optional rocking sole;

FIG. 14 is an environmental view of the orthotic of FIG. 1 showing a leg orthotic with optional rocker sole and optional balancer;

FIG. 15 perspective view of the optional balancer of FIG. 14;

FIG. 16 is a perspective view of the orthotic of FIGS. 1 and 14 showing an optional sport protector and sport balancer;

FIG. 17 is a perspective view of the orthotic leg brace of FIG. 16;

FIG. 18 is an environmental view of the orthotic of FIG. 1 showing an optional second orthotic;

FIG. 19 is an environmental view of the orthotics of FIGS. 1 and 18 showing an optional waist and hip support girdle;

FIG. 20 is an environmental view of the orthotics of FIGS. 1 and 18 showing an optional thoracic girdle;

FIG. 21 is an environmental view of the orthotic of FIG. 20 showing an optional axillary extension;

FIG. 22 is a perspective view of an embodiment of the leg orthotic system showing a padded leg sleeve;

FIG. 23 is a perspective view of the leg orthotic system of FIG. 22 showing selective openings for applying the sleeve to a user's leg;

FIG. 24 is a perspective view of an embodiment of the leg orthotic system showing optional knee unweighting.

### DETAILED DESCRIPTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present there between. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section.

As used herein, the singular forms “a,” “an,” and “the,” are intended to include the plural forms as well, unless the context clearly indicates otherwise. It will be further understood that the terms “comprises” and/or “comprising,” “includes” and/or “including,” and “have” and/or “having,” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom,” and “upper” or “top,” “inner” or “outer,” and “medial” or “lateral” may be used herein to describe one element's relationship to other elements as illustrated in the Figures. It will be understood that relative terms are intended to encompass different orientations of the device in addition to the orientation depicted in the Figures.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing.

Embodiments of the disclosure exemplify a non-weight bearing leg orthotic. While the detailed description is based upon the following description and drawings depicting an illustrated size and shape, the non-weight bearing leg orthotic may be provided in any size, shape, and dimension. Elements referred to in the disclosure may be packaged with and comprised of various materials described hereafter.

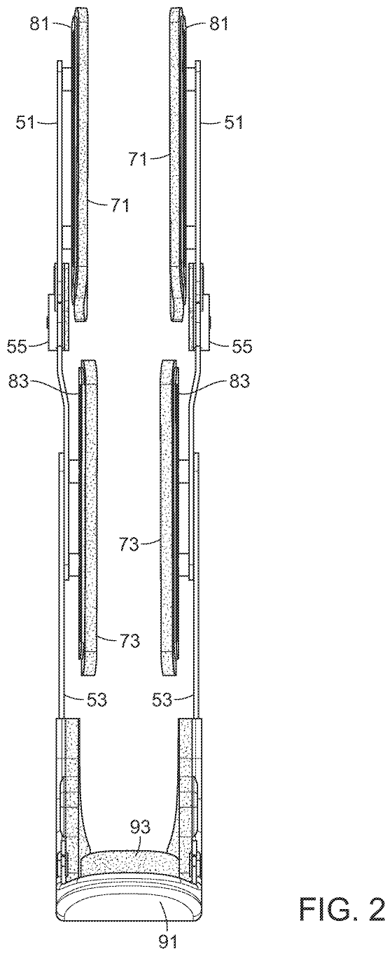

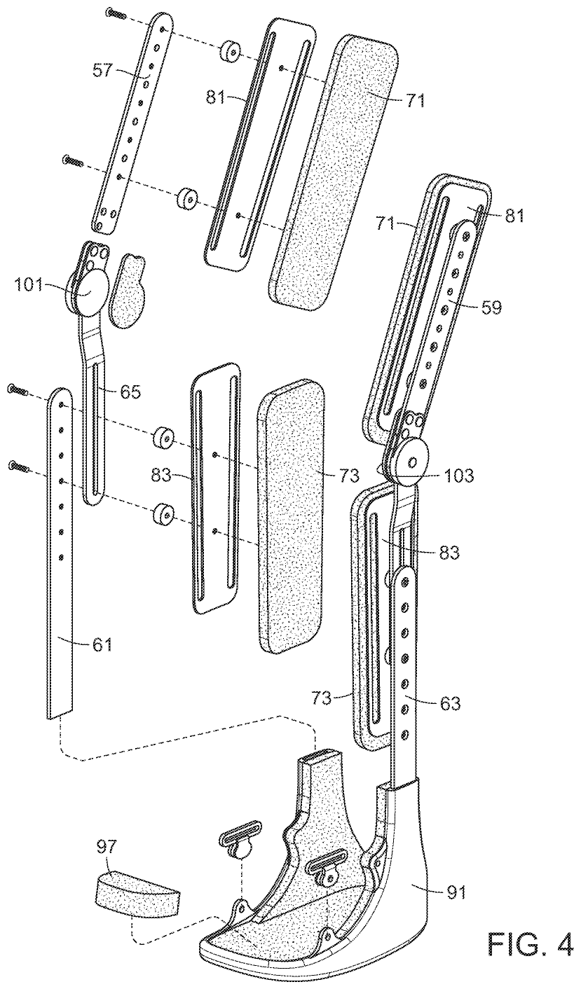

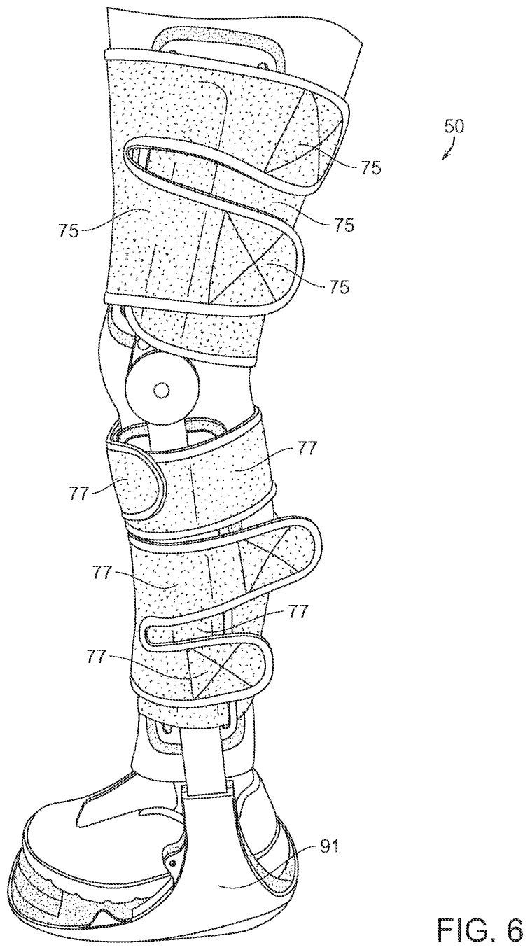

As shown in FIG. 6 an assembled embodiment of the disclosure teaching an unweighting leg orthotic (ULO) is generally referred to by reference character 50. As shown in FIG. 2 a ULO 50 may include a set of upper supports 51 hingedly connected to a set of lower supports 53 by a set of joints 55 that may rotate freely consistent with a user's natural range of motion. As shown in FIG. 4 said set of upper supports 51 may comprise an upper medial member 57 and an upper lateral member 59. Said set of lower supports 53 may comprise a lower medial member 61 and a lower lateral member 63.

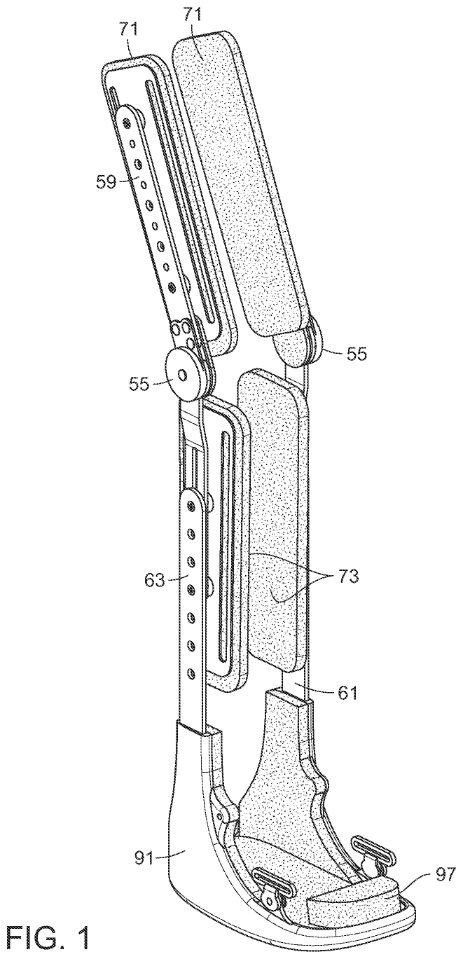

Said set of upper supports 51 and said set of lower supports 53 provide framework to which a user's weight is distributed. Each may be comprised of plastics, composite plastics, metallics or metallic alloys. As shown in FIG. 1, said set of joints 55 couple said set of upper supports 51 to said set of lower supports 53. Preferably this coupling is in a hinged or jointed composition providing a user with a safe range of motion along the knee. Other embodiments may have ball and socket type joints that allow greater range of motion to the user. Said set of joints 55 may be comprised of the same materials as the set of upper supports 51 and said set of lower supports 53. The ULO 50 may be assembled as shown in FIG. 4 according to methods known within the art.

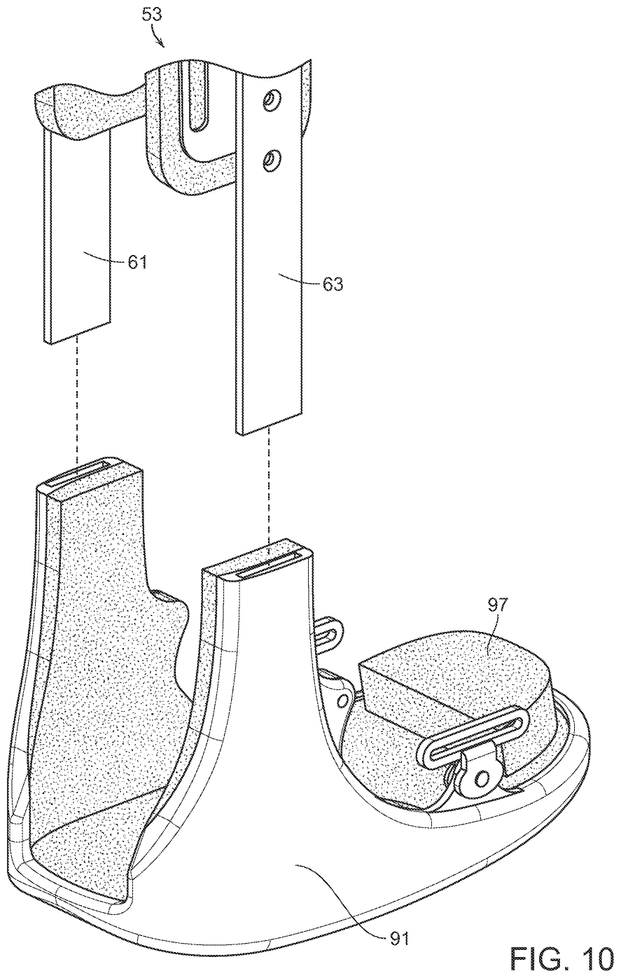

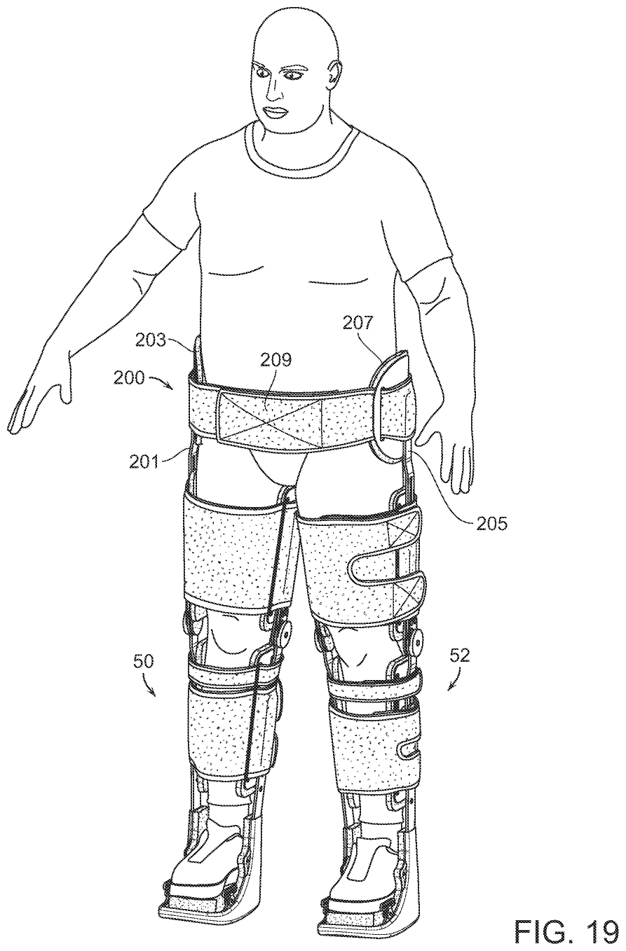

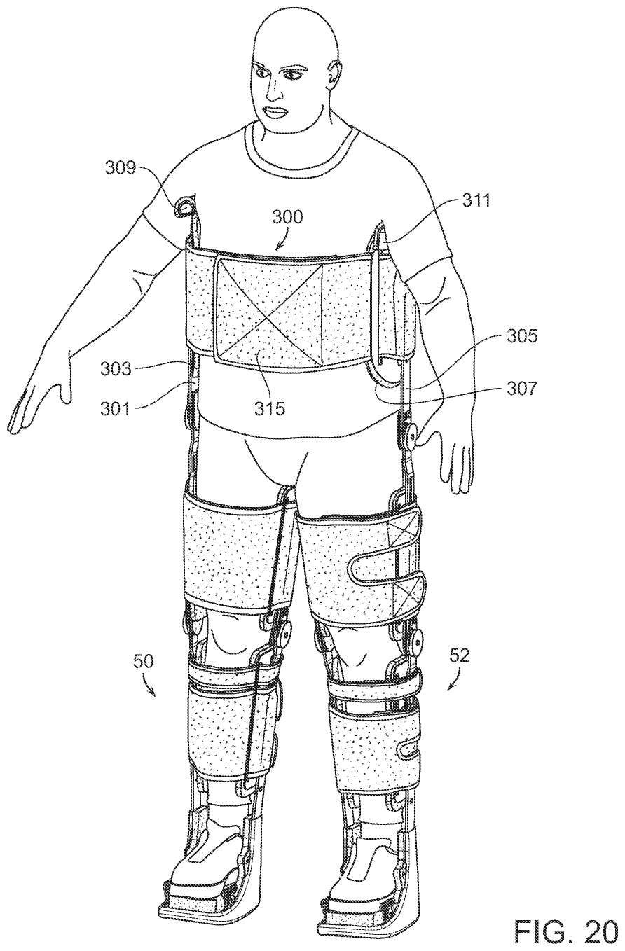

Said ULO 50 may accommodate various joints about the user. Couplings may be jointed as optionally demonstrated by said set of joints 55 as shown in FIG. 1. Couplings may be optionally configured for fixed range of motion as shown by said lower medial member 61 and said lower lateral member 63 as shown in FIG. 10. Couplings across embodiments of the disclosure may be of a fixed range of motion or variable range of motion, including those depicted in FIGS. 19 and 20.

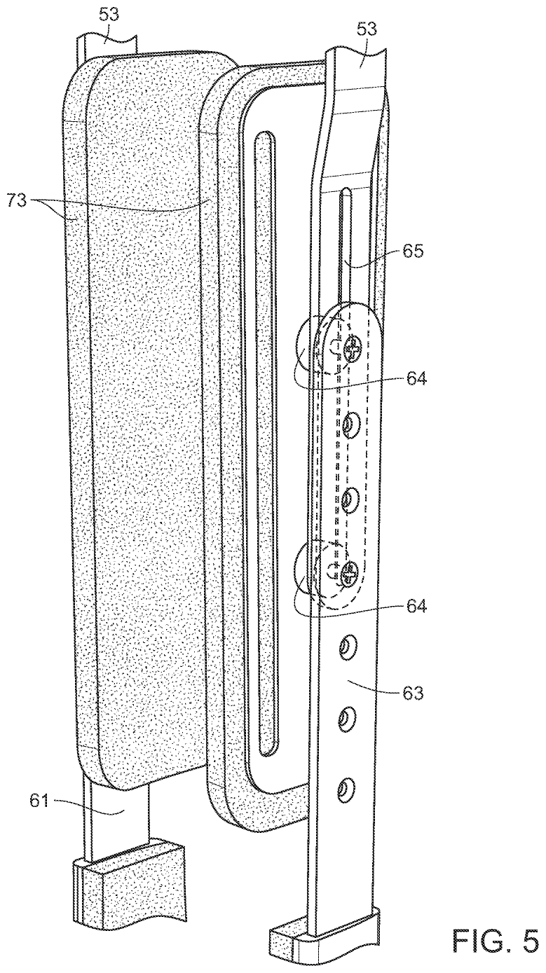

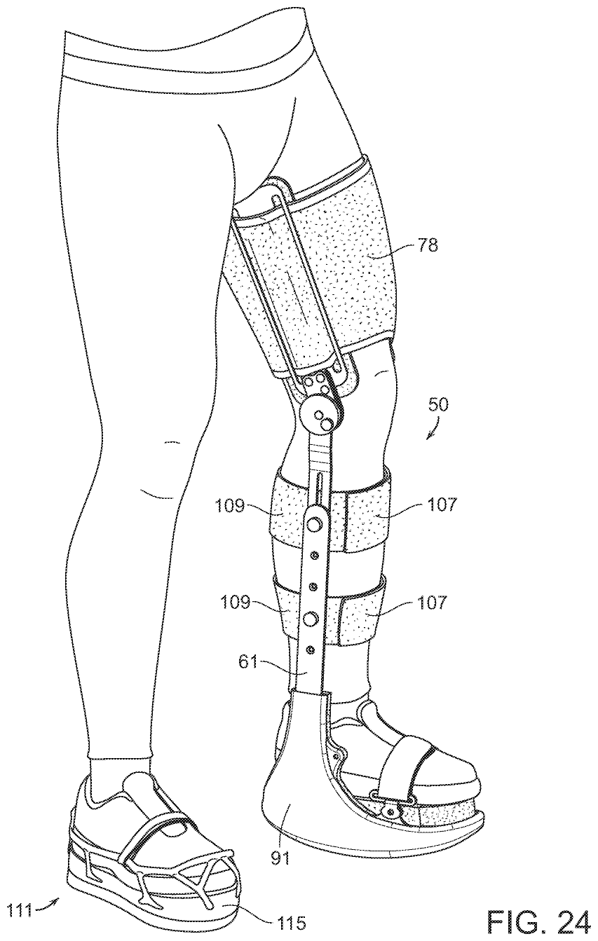

Total weight offloading is preferably achieved when said set of joints 55 are unlocked, as well as the upper supports 51 and lower supports 53 are secured to the corresponding upper and lower portions of the user's leg. As shown in FIG. 24 said set of joints 55 may provide optional locking capability affording additional stabilization options to a user. In such configurations a user may customize the range of motion permitted by said set of joints 55 thereby tailoring the degree to which the knee joint may hinge. When locked, said set of joints 55 provides additional elements through which the user's weight may be transferred from the user's thigh to the ground, with the lower supports 53 not being secured to the corresponding lower portion of the user's leg, as discussed more fully below, so as to eliminate any weight bearing on the user's foot and knee. As shown in FIG. 5, said unweighting leg orthotic 50 (ULO) may optionally comprise a lower adjuster 64 along said lower medial member 61, said lower lateral member 63, and accommodated by said set of joints 55. Said lower adjuster 64 may be comprised of a groove 65 and replaceable fasteners allowing said lower adjuster 64 to slidingly associate with said lower medial member 61 and said lower lateral member 63. The sliding association may alter the length of the ULO 50. This adjustment permits operators of various heights to equip said ULO 50. Said lower adjuster 64 may be comprised of the same materials as said set of upper supports 51 and said set of lower supports 53.

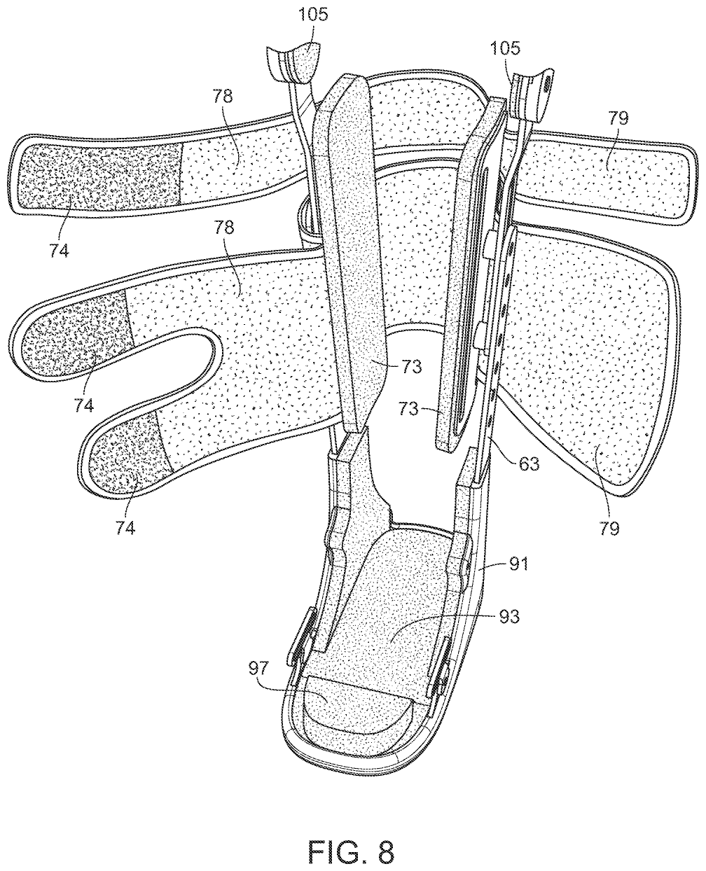

As shown in FIG. 4, said set of joints 55 may be further comprised of a medial joint face 101 and lateral joint face 103. The medial joint face 101 and lateral joint face 103 are oriented toward each other and located at the hinge of each of said set of joints 55. As shown in FIG. 8 a set of joint pads 105 may abut said medial joint face 101 and said lateral joint face 103 as shown in FIGS. 2 and 4. Said set of joint pads 105 provide cushioning to an operator and serve to set various supports apart from various parts of a user's leg.

Said set of joints 55 may be comprised of the same materials as said set of upper supports 51 and said set of lower supports 53 and may also include plastics and ceramics. Said set of joint pads 105 may be comprised of rubber, plastics, neoprene, and composites thereof as described by sleeve materials above.

Said unweighting leg orthotic (ULO) 50 may further comprise a plurality of upper brackets 81 and a plurality of lower brackets 83 as shown in FIGS. 1, 2, and 4. Said plurality of upper brackets 81 is deep to said plurality of upper supports 51 as shown in FIG. 2. Said plurality of lower brackets 83 is deep to said plurality of lower supports 53 as also shown in FIG. 2.

Said plurality of upper brackets 81 and said plurality of lower brackets 83 are fixed to said supports by means known with the art. They each serve as an interface for wraps providing an entry and exit for the same. Said plurality of upper brackets 81 and said plurality of lower brackets 83 may be made of the same materials as said set of upper supports 51 and said set of lower supports 53.

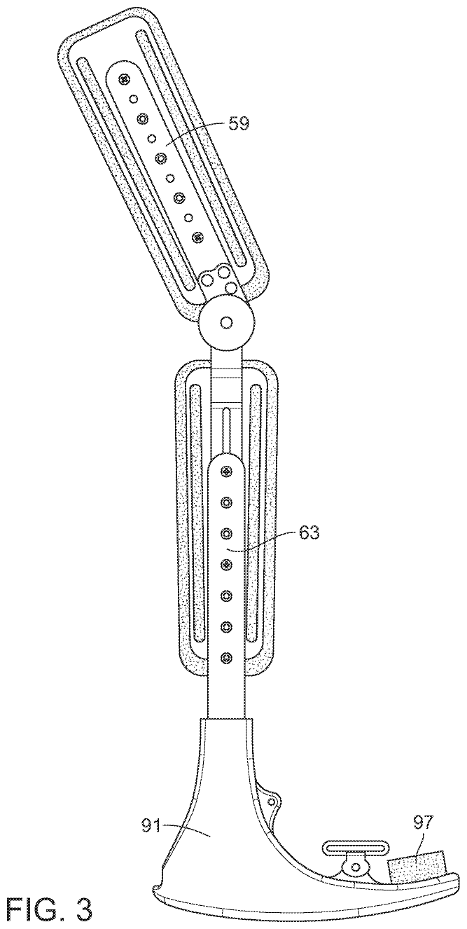

Said unweighting leg orthotic (ULO) 50 may further comprise a plurality of upper pads 71 and a plurality of lower pads 73 as shown in FIGS. 1-4. Said plurality of upper pads 71 is deep to said plurality of upper brackets 81 and said plurality of lower pads 73 is deep to said plurality of lower brackets 83. The various pads are connected to the various supports by means known within the art.

Said various pads 71, 73 cushion and support a user's leg as said ULO 50 transfers weight from a user's lower limbs to said plurality of upper supports 51 and said plurality of lower supports 53. The pads 71, 73 provide further functionality by foregoing the need for protective sleeves or padding underneath the ULO 50. Said plurality of upper pads 71 and said plurality of lower pads 73 may be comprised of the same materials as said set of joint pads 105. When properly and securely held, the upper and lower pads 71, 73 are designed to carry the full weight of a user that would ordinarily be transferred through the bottom of a user's foot.

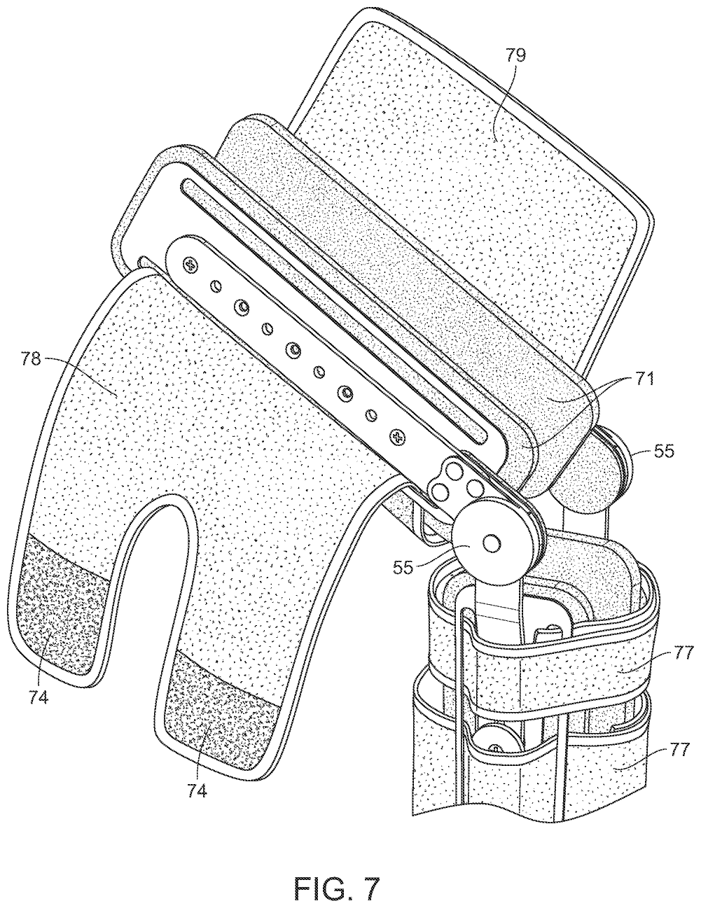

As shown in FIG. 6, said unweighting leg orthotic (ULO) 50 may further comprise a plurality of upper wraps 75 and a plurality of lower wraps 77. As shown in FIGS. 7 and 8 each said plurality of wraps may further comprise a medial wrap end 78 and an opposite lateral wrap end 79. Each wrap end may provide a fastener 74, including as hook and loop, snaps, or buttons, to the opposite wrap end.

In preferred embodiments said medial wrap end 78 comprises the fastener 74 for binding to said lateral wrap end 79 as shown in FIGS. 7 and 8. A user may insert such exemplary wraps through said plurality of upper brackets 81 or said plurality of lower brackets 83 as shown particularly in FIG. 8. Said lateral wrap end 79 may be disposed over the user's limb. Then said medial wrap end 78 may be disposed over said lateral wrap end 79 and fixed by said fastener 74. In other embodiments said fastener 74 may instead be positioned on said lateral wrap end 79 which fixes to an underlying medial wrap end 78. In any embodiment comforting materials may line said plurality of upper wraps 75 or said plurality of lower wraps 77.

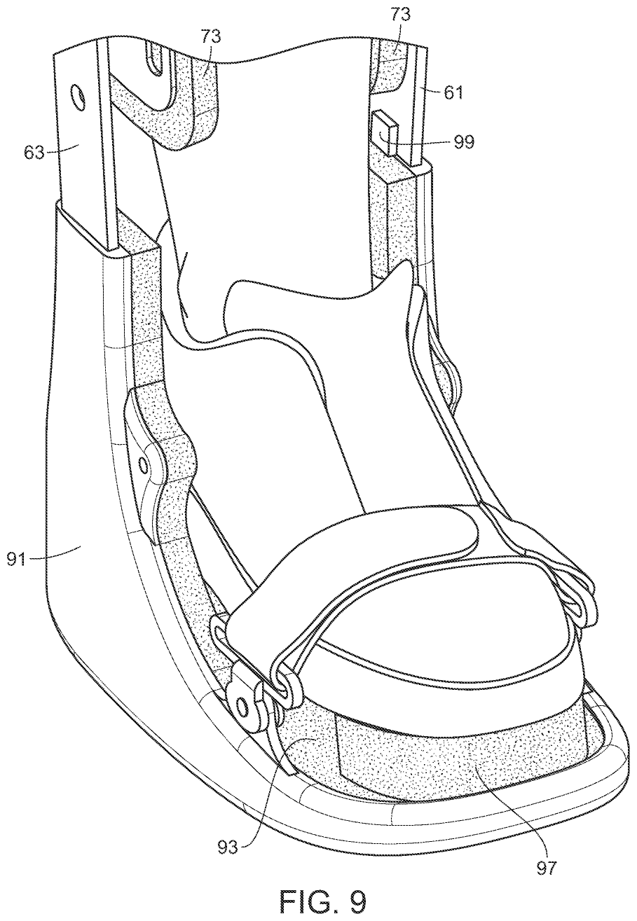

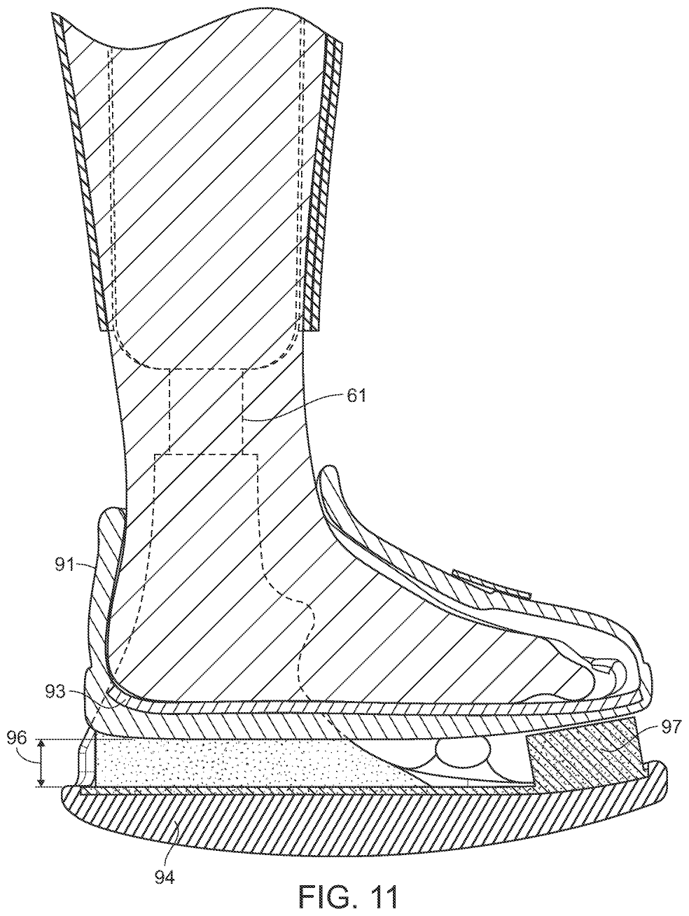

Said unweighting leg orthotic (ULO) 50 may further comprise a pedal protector 91 as shown in FIGS. 9-13. Said pedal protector 91 may comprise an insole 93 and a toe pad 97 defining a void 95 across which the user's foot does not contact the insole 93 as shown in FIG. 11. Said void 95 may be akin to a gap, or space. Said toe pad 97 maintains ankle flexion and may optionally be removed where flexion is not desired. Said pedal protector 91 may be configured to allow for traditional footwear to be used inside said ULO 50 as shown in FIG. 9.

As shown in FIG. 6, said void 95 is enabled by virtue of said plurality of upper wraps 75 and said plurality of lower wraps 77 transferring a user's weight to said set of upper supports 51 and said set of lower supports 53. The void 95 allows users the benefits of limb bracing systems that enable standing and ambulatory movement without requiring the bottoms of the user's feet to be in contact with another surface, i.e., the insole 93.

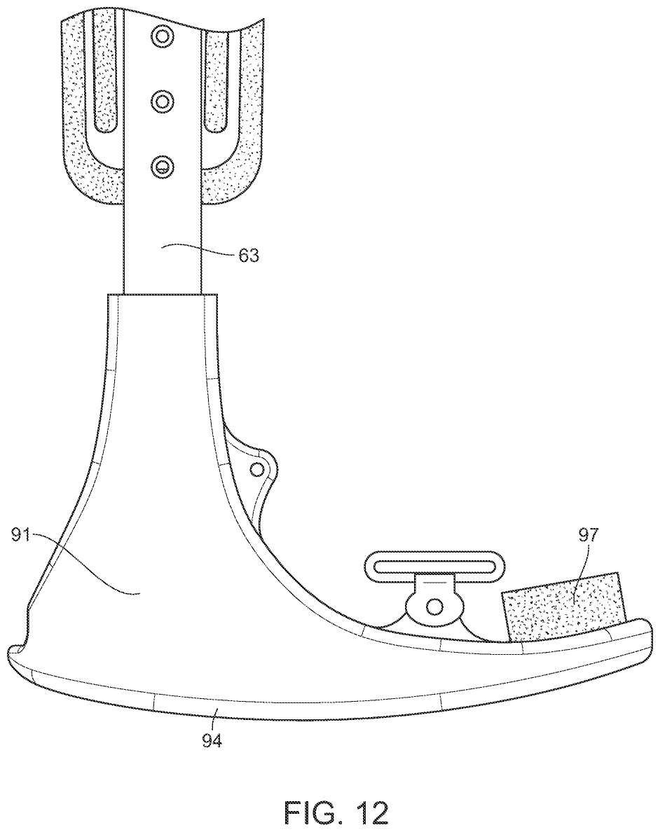

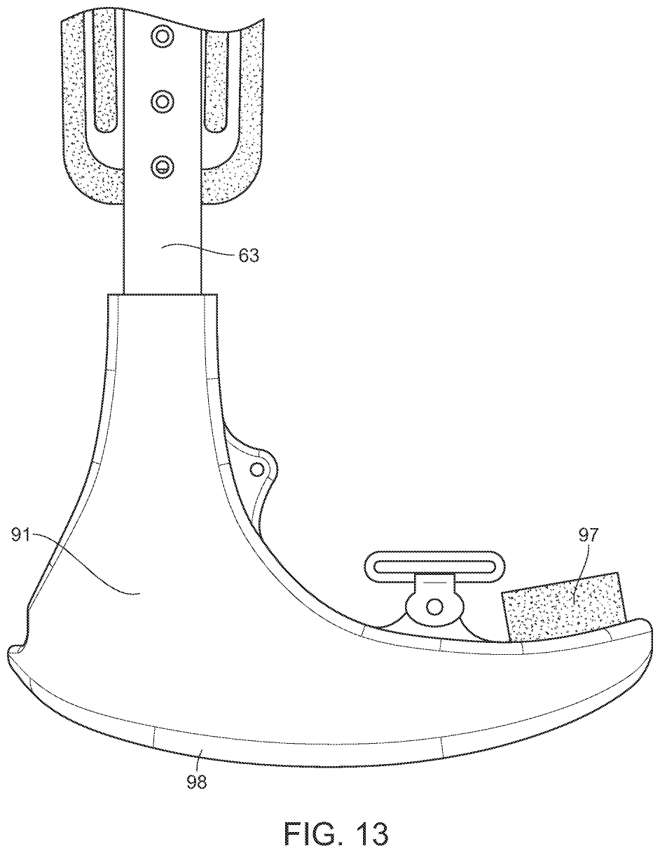

Said pedal protector 91 may be removably and adjustably coupled to said set of lower supports 53 as shown in FIG. 10. Said lower medial member 61 and said lower lateral member 63 insertingly associate with said pedal protector 91. Said pedal protector 91 may be worn over clothing and configured for an optional flat bottom 94 as shown in FIG. 12 or a rocker bottom 98 as shown in FIG. 13 or any shaped combination thereof.

In other embodiments, said pedal protector 91 may be removably coupled by a quick release 99 as shown in FIG. 9. Said quick release 99 facilitates rapid donning and doffing of said pedal protector 91 without removing other elements of said ULO 50. Said quick release 99 may be preferably configured for depressible release or attached otherwise including means such as straps or hook and loop.

Other embodiments of the disclosure provide a ULO 50 with partial weight bearing capability through the foot. Weight may be distributed through any part of the foot, preferably through the sole or heel. Any percentage of the user's weight may be distributed as such by modifying the angle of said set of joints 55, dimension of said gap 96, tightness of said plurality of upper wraps 75, plurality of lower wraps 77, and any combination thereof.

In other embodiments said pedal protector 91 may have an optional configuration achieved by removing said insole 93, and any of said flat bottom 94 or said rocker bottom 98, leaving only medial and lateral pedal frames providing walking edges for contact with the ground. The walking edges preferably extend below the sole of the user's foot so as to create the gap 96 for complete offloading or weight percentage offloading as discussed. At least some and preferably all padding, except for said toe pad 97 may be removed. Support of said toe pad 97 may be maintained by a linear support proximate to the toe area of the pedal protector 91. This configuration allows for a light-weight or more open design which greater facilitates locomotion and comfort without foregoing support.

As shown in FIG. 24, one or more of the lower members 61/63, lower pads 73, lower brackets 83, and lower wraps 77 may be eliminated from the lower supports 53, such that the set of lower supports 53. In this form, the set of lower supports 53 are not secured to the user's lower leg, such that the same is permitted to hinge freely at the set of joints 55. In such configuration, the lower supports 53 may include loose-fitting anterior bands 107 and posterior bands 109 so as to allow the user's lower leg greater freedom of movement while still generally restricting range of motion to a safe range in the region between the set of lower supports 53 during the function of walking on the unweighting leg orthotic 50. Alternatively, this configuration may include one or more of the lower members 61/63, lower pads 73, lower brackets 83, and lower wraps 77 provided that the same are not secured tightly to the user's lower leg. When combined with the above-mentioned optional locking mechanism in the set of joints 55, this configuration may unweight both the user's foot and knee.

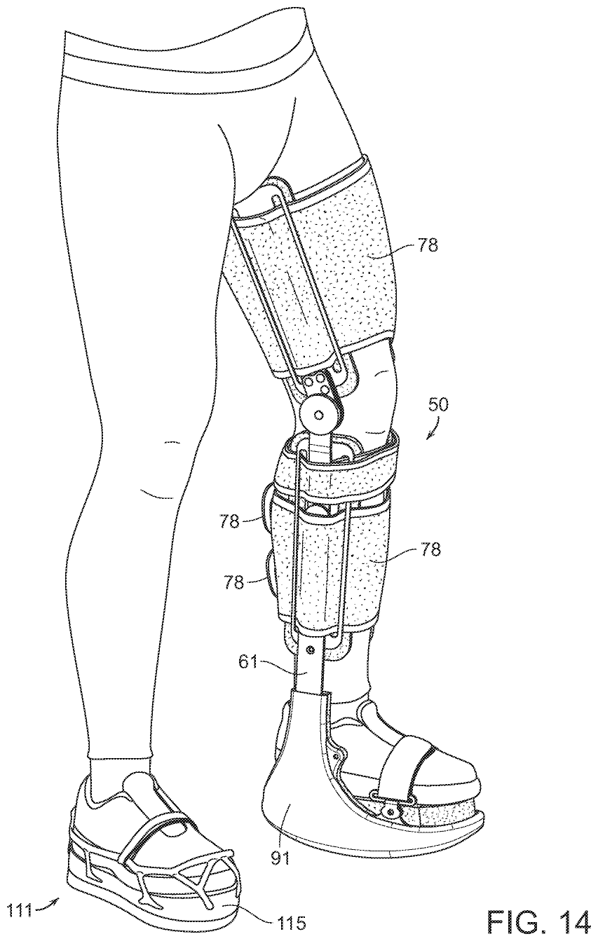

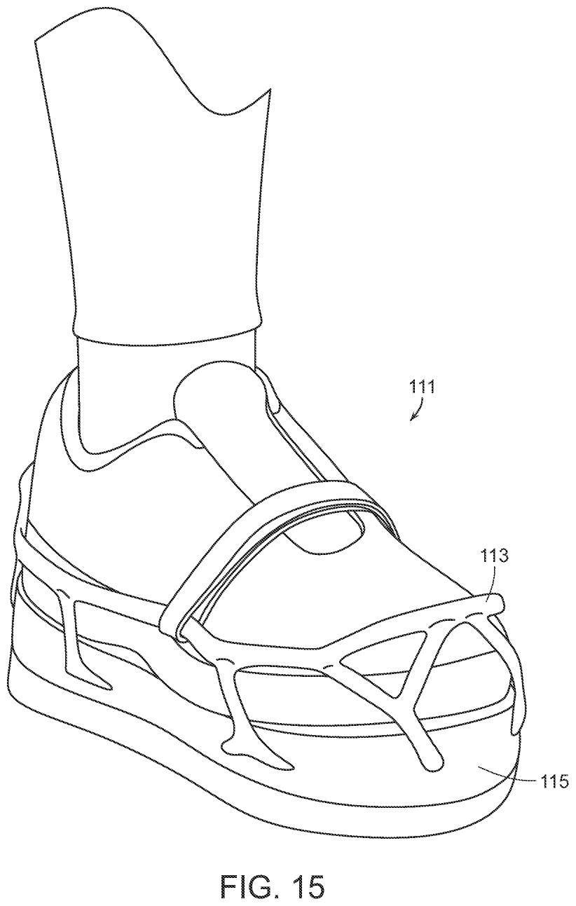

As shown in FIGS. 14 and 15, a system incorporating said unweighting leg orthotic (ULO) 50 may further comprise a balancer 111 designed for a user's opposite foot. Said balancer 111 provides a user the ability to offset additional leg length gained by deploying said ULO 50 on one leg. Said balancer 111 may be worn on a foot opposite to the leg brace alone or over a user's preferred footwear.

As shown in FIG. 15, said balancer 111 may comprise a shoe 113 which contains an insert 115. Said insert may be of any height configurable for use with a ULO 50. Ideally, the combined height of the shoe 113 and insert 115 of the balancer 111 is consistent with or equal to the combined height of the sole 93 and a dimension of the gap 96 of the pedal protector 91 with the purpose of balancing the user's leg lengths to effective standing surfaces.

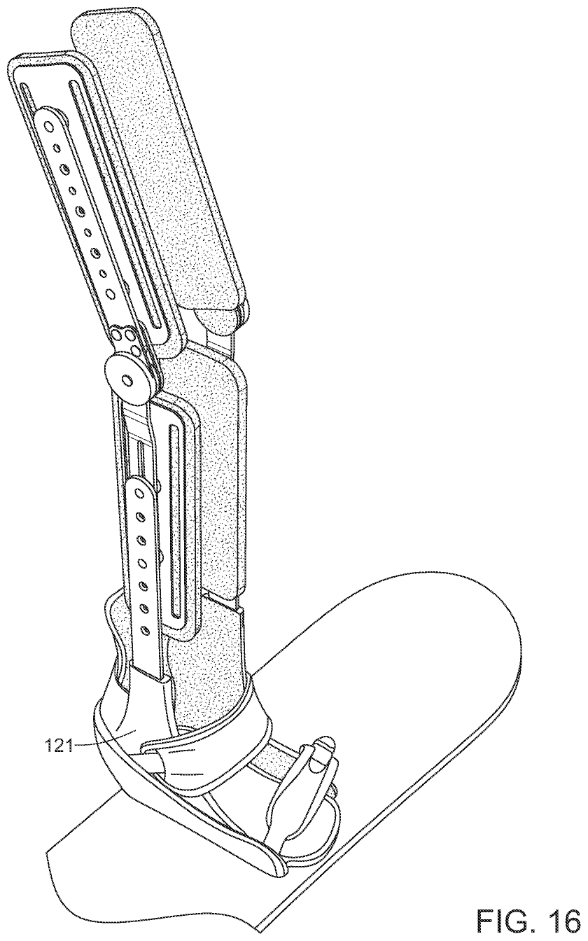

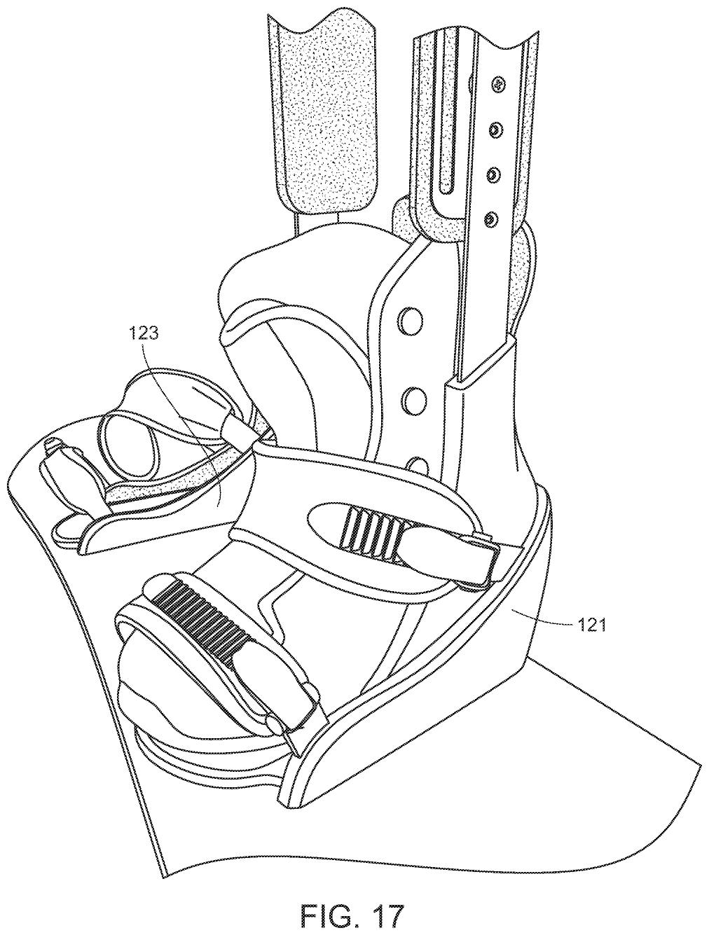

Other embodiments of the disclosure for an unweighting leg orthotic (ULO) 50 may comprise a sport protector 121 and sport balancer 123 as shown in FIGS. 16 and 17 which may correspond to said pedal protector 91 and said balancer 111 as shown in FIG. 14. Said sport protector 121 may be configured for any boot adaptor including a snowboard boot adaptor or ski boot adaptor. Said sport protector 121 may be configured for use with any recreational board where fasteners may be placed included skateboards and surf boards. Said sport balancer 123 may be similarly configured and provide similar benefits as said balancer 111.

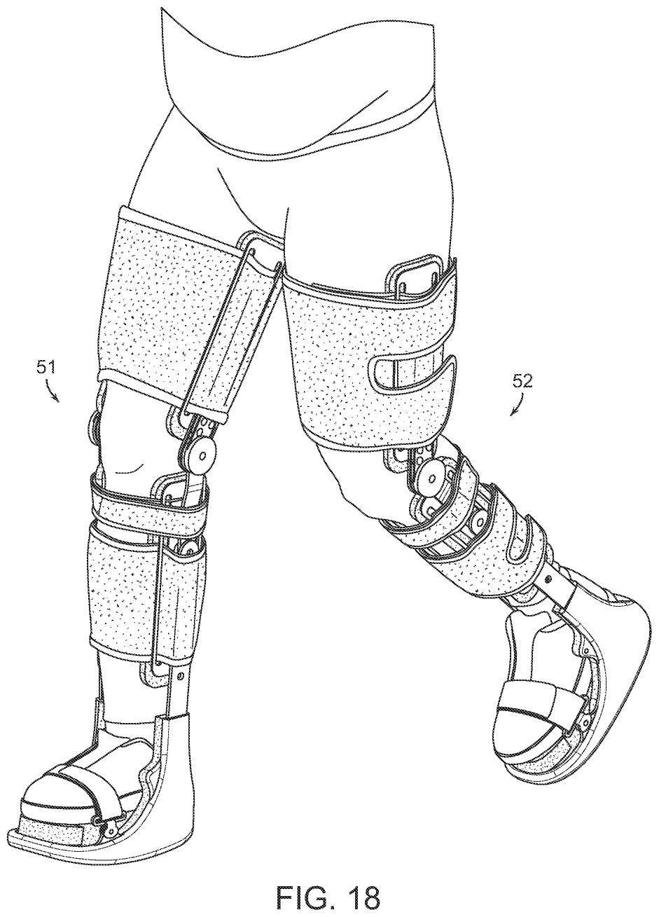

A user may use embodiments of said unweighting limb orthotic (ULO) 50 in tandem using a second unweighted leg orthotic 52 as shown in FIG. 18. Said second leg orthotic 52 may be equivalent to said unweighted leg orthotic 50. Other embodiments of the present disclosure may comprise a waist girdle 200 for use with such configurations as shown in FIG. 19. Said waist girdle 200 may comprise a first hip extension 201 and a first hip pad 203 and a second hip extension 205 and a second hip pad 207. A waist band 209 may interface with each extension and pad transversely across the user. All pads may be either fixed or removably attached for convenient replacement. Said first hip extension 201 and second hip extension 205 are hingedly coupled to each upper lateral member 61 of each set of upper supports 51 proximate to the corresponding hip joints of the user. The coupling provides a user with additional waist support. Each coupling is secured by means known within the art.

Said first hip extension 201 may be coupled with said first hip pad 203. Said second hip extension 205 is coupled with said second hip pad 207. Said first hip pad 203 and said second hip pad 207 insulate the user from the structural extensions. The extensions may be comprised of the same materials as said set of upper supports 51 and said set of lower supports 53.

Said first hip pad 203 and said second hip pad 207 may have openings that allow a waist band 209 to associate with each hip extension as shown in FIG. 19 in similar fashion as does each plurality of wraps associate with each bracket as shown in FIG. 8. Said waist band 209 allows for a greater percentage of bodyweight to be transferred to said set of upper supports 51 and said set of lower supports 53 than do tandem ULOs 50 alone without said waist girdle 200.

Said unweighting leg orthotic (ULO) 50 in tandem may further comprise a thoracic girdle 300 as shown in FIG. 20. Said thoracic girdle 300 may comprise a first thoracic extension 301 and a first thoracic pad 303 and a second thoracic extension 305 and a second thoracic pad 307. A thoracic band 315 band may interface with each extension and pad and transversely span across the user.

Said first thoracic extension 301 and second thoracic extension 305 hingedly associate with each said upper lateral member 61 of said set of upper supports 51. The hinged association provides a user with greater waist mobility.

Said first thoracic extension 301 is coupled with said first thoracic pad 303. Said second thoracic extension 305 is coupled with said second thoracic pad 307. Said first thoracic pad 303 and said second thoracic pad 307 insulate the user from the structural extensions. The extensions may be comprised of the same materials as said set of upper supports 51 and said set of lower supports 53.

Said first thoracic pad 303 and said second thoracic pad 307 may have openings that allow a thoracic band 315 to associate with each thoracic extension as shown in FIG. 20 in similar fashion as does each plurality of wraps associate with each bracket as shown in FIG. 8. Said thoracic band 315 enables a greater percentage of bodyweight to be transferred to said set of upper supports 51 and said set of lower supports 53 than do tandem ULOs 50 alone without said thoracic girdle 200.

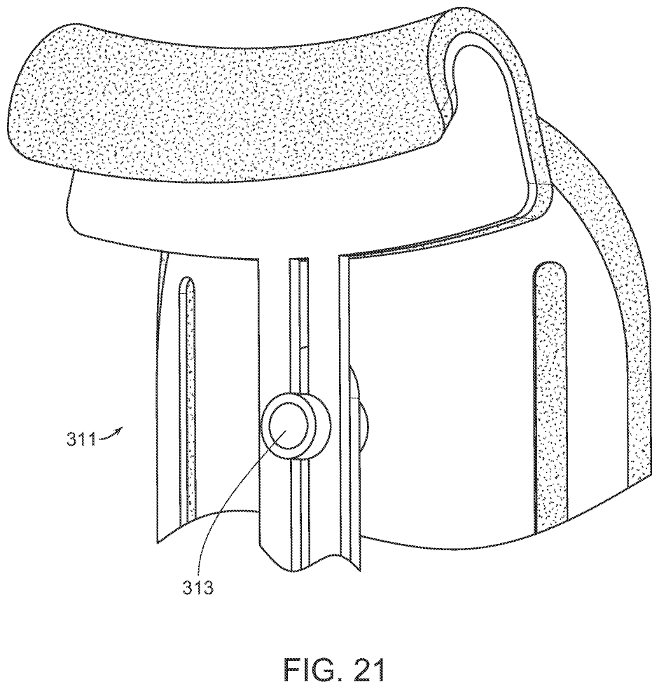

In other embodiments, said thoracic girdle 300 may further comprise a first axillary support 309 and a second axillary support 311 as shown in FIGS. 20 and 21. Each axillary support may have an axillary adjustment 313 enabling ULO 50 use by operators of various heights. Said first axillary support 309 is coupled to said first thoracic extension 301 and said second axillary support 311 is coupled to said second thoracic extension 305.

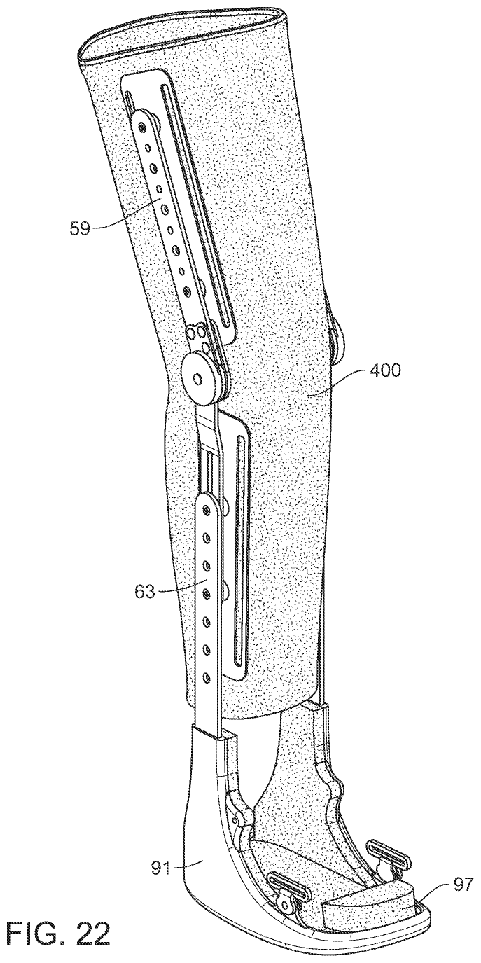

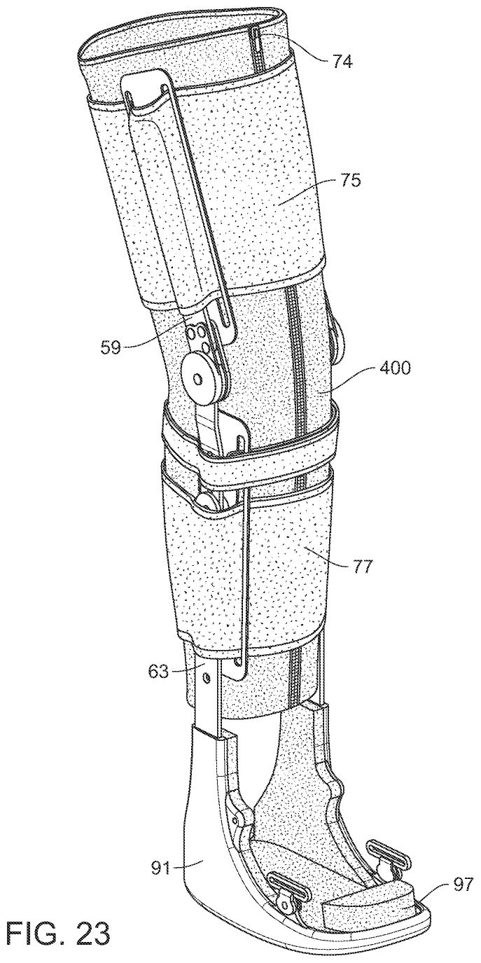

FIGS. 22 and 23 show an alternate embodiment of the ULO 50 wherein said plurality of upper brackets 81, plurality of lower brackets 83, plurality of upper pads 71, plurality of lower pads 73 and plurality of upper wraps 75, and plurality of lower wraps 77 are replaced with a leg sleeve 400 configured to span the user's leg from proximate to the hip joint to proximate to the ankle joint. The leg sleeve 400 is configured as or similar to that of a wetsuit such that it closely or snugly conforms to the user's leg. The leg sleeve 400 is preferably made from neoprene or similar wetsuit material such that it breathes or otherwise allows for proper ventilation or cooling.

The leg sleeve 400 is attached along its length to the corresponding upper medial member 57, upper lateral member 59, lower medial member 61, and lower lateral member 63 and is configured to receive a user's leg as a pant leg or similar covering. When properly configured, the leg sleeve 400 will fully support the user and transfer the weight to the upper medial member 57, upper lateral member 59, and lower medial member 61 and lower lateral member 63.

The leg sleeve 400 may be used in conjunction with said plurality of upper wraps 75 and said plurality of lower wraps 77 to ensure a more secure fit. In an alternate embodiment, the leg sleeve 400 may have a longitudinal opening spanning from top to bottom, such that a user may wrap the leg sleeve 400 around his/her leg rather than insert the leg from the top. The longitudinal opening may include a said fastener 74 such as a zipper or hook and loop for closing the leg sleeve 400 around the user's leg. Although several embodiments have been described in detail for purposes of illustration, various modifications may be made without departing from the scope and spirit of the invention.

1. An unweighting leg orthotic system, comprising:
a leg brace comprising a set of upper supports hingedly coupled to a set of lower supports at a set of joints, said leg brace configured for removable attachment to a user's leg;
an upper medial member and an upper lateral member associated with said set of upper supports, and a lower medial member and a lower lateral member associated with said set of lower supports;
a plurality of upper wraps engaging with the upper medial member and upper lateral member, and configured so as to removably hold the upper medial member and upper lateral member securely against a corresponding medial face and lateral face of the user's leg;
a plurality of lower wraps engaging with the lower medial member and lower lateral member, and configured so as to removably hold the lower medial member and lower lateral member securely against a corresponding medial face and lateral face of the user's leg; and
a pedal protector having an insole, wherein the pedal protector is removably connected to said set of lower supports, wherein said pedal protector is configured so as to maintain a gap between the insole of the pedal protector and a sole of the user's foot.

2. The unweighting leg orthotic system of claim 1, said pedal protector further comprising a toe bolster on the insole of the pedal protector configured so as to support toes of the user's foot.

3. The unweighting leg orthotic system of claim 1, further comprising a quick release interface between said set of lower supports and said pedal protector.

4. The unweighting leg orthotic system of claim 1, wherein said set of lower supports are adjustable in length so as to make a dimension of the gap adjustable.

5. The unweighting leg orthotic system of claim 1, wherein said plurality of upper wraps and said plurality of lower wraps each comprise a medial wrap end, a lateral wrap end, and a fastener configured to removably connect said lateral wrap end to said medial wrap end.

6. The unweighting leg orthotic system of claim 1, wherein said set of joints further comprise a medial joint face, a lateral joint face, and a set of joint pads associated therewith configured so as to abut against the user's knee.

7. The unweighting leg orthotic system of claim 1, wherein said pedal protector comprises a boot compatible with snowboard bindings, ski bindings, waterski bindings, skateboard bindings, or scooter bindings.

8. The unweighting leg orthotic system of claim 1, further comprising a pedal balancer configured for use on a user's foot opposite the leg brace, wherein the pedal balancer has a sole thickness equal in dimension to a combined dimension of the gap and a thickness of the insole of the pedal protector.

9. The unweighting leg orthotic system of claim 1, further comprising a plurality of upper pads associated with the upper medial member and upper lateral member, wherein the plurality of upper pads are configured so as to abut a substantial length of the corresponding medial face and lateral face of an upper portion of the user's leg.

10. The unweighting leg orthotic system of claim 1, further comprising a plurality of lower pads associated with the lower medial member and lower lateral member, wherein the plurality of lower pads are configured so as to abut a substantial length of the corresponding medial face and lateral face of a lower portion of the user's leg.

11. A unweighting leg orthotic system, comprising:
a first leg brace and a second leg brace, each leg brace comprising a set of upper supports hingedly coupled to a set of lower supports at a set of joints, the first leg brace and the second leg brace each configured for removable attachment to a user's corresponding leg;
wherein each leg brace comprises:
an upper medial member and an upper lateral member associated with said set of upper supports, and a lower medial member and a lower lateral member associated with said set of lower supports;
a plurality of upper pads associated with the upper medial member and upper lateral member, and a plurality of lower pads associated with the lower medial member and lower lateral member;
a plurality of upper wraps engaging with the upper medial member and upper lateral member, and configured to removably hold the plurality of upper pads securely against a corresponding medial face and lateral face of the user's corresponding leg;
a plurality of lower wraps engaging with the lower medial member and lower lateral member, and configured to removably hold the plurality of lower pads securely against a corresponding medial face and lateral face of the user's corresponding leg; and
a pedal protector having an insole, wherein the pedal protector is removably connected to said set of lower supports, wherein said pedal protector is configured so as to maintain a gap between the insole of the pedal protector and a sole of the user's corresponding foot.

12. The unweighting leg orthotic system of claim 11, further comprising:
a first hip extension associated with the upper lateral member of the first leg brace and a second hip extension associated with the upper lateral member of the second leg brace;
a first hip pad associated with the first hip extension and a second hip pad associated with the second hip extension; and
a waist band associated with the first hip extension and the second hip extension, wherein the waist band is configured to transverse a user's waist and to removably hold the first hip pad and second hip pad securely against corresponding lateral faces of the user's waist.

13. The unweighting leg orthotic system of claim 12, further comprising:
a first thoracic extension associated with the first hip extension and a second thoracic extension associated with the second hip extension; and
a thoracic girdle engaging with the first thoracic extension and the second thoracic extension, and configured so as to generally transverse a user's torso and removably hold the first thoracic extension and the second thoracic extension securely against corresponding lateral sides of the user's torso.

14. The unweighting leg orthotic system of claim 13, further comprising a first thoracic pad coupled to said first thoracic extension, and a second thoracic pad coupled to said second thoracic extension.

15. The unweighting leg orthotic system of claim 13, further comprising a first axillary support coupled to said first thoracic extension and a second axillary support coupled to said second thoracic extension.

16. The unweighting leg orthotic system of claim 15 wherein said first axillary support and said second axillary support are each adjustable relative to the corresponding first thoracic extension and second thoracic extension.

17. The unweighting leg orthotic system of claim 11, further comprising a plurality of upper pads associated with the upper medial member and upper lateral member, wherein the plurality of upper pads are configured so as to abut a substantial length of the corresponding medial face and lateral face of an upper portion of the user's corresponding leg.

18. The unweighting leg orthotic system of claim 11, further comprising a plurality of lower pads associated with the lower medial member and lower lateral member, wherein the plurality of lower pads are configured so as to abut a substantial length of the corresponding medial face and lateral face of a lower portion of the user's corresponding leg.

19. The unweighting leg orthotic system of claim 1, wherein the plurality of upper wraps engaging with the upper medial member and upper lateral member and the plurality of lower wraps engaging with the lower medial member and lower lateral member are all configured to support substantially all of a user's weight in the leg brace when removably held against the corresponding medial face and lateral face.

20. The unweighting leg orthotic system of claim 11, wherein the plurality of upper wraps engaging with the upper medial member and upper lateral member and the plurality of lower wraps engaging with the lower medial member and lower lateral member, on each of the first leg brace and the second leg brace, are all configured to support substantially all of a user's weight in the first leg brace and the second leg brace when removably held against the corresponding medial face and lateral face.
